(19) United States
(12) Patent Application Publication
(10) Pub. No.: US 2026/0183080 A1
(43) Pub. Date: Jul. 2, 2026

(54) RADIOPAQUE ANATOMY MARKERS AND DELIVERY SYSTEM

(71) Applicant: Medtronic, Inc., Minneapolis, MN (US)

(72) Inventor: Donna CURLEY, Galway (IE)

(21) Appl. No.: 19/129,370

(22) PCT Filed: Nov. 10, 2023
(86) PCT No.: PCT/IB2023/061378

§ 371 Date: May 13, 2025

Related U.S. Application Data
(60) Provisional application No. 63/384,447, filed on Nov. 21, 2022.

(51) Int. Cl. A61B 90/00; A61M 25/00; A61M 25/01

(52) U.S. Cl. CPC A61B 90/39; A61B 2090/3966; A61B 2090/3987; A61M 25/0068; A61M 25/0141; A61M 25/0158; A61M 2210/125

(57) ABSTRACT

A system for implanting a radiopaque marker includes a catheter and a radiopaque marker disposed within the catheter. The catheter includes a delivery configuration and an implantation configuration. The catheter further includes a first shaft configured to curl when the catheter is in the implantation configuration, the first shaft including a first opening positioned at a side of the first shaft, a second shaft positioned within the first shaft and configured to transition the catheter between the delivery configuration and the implantation configuration, the second shaft including a second opening, and a pusher shaft positioned within the second shaft. The radiopaque is disposed within the second shaft with the catheter in the delivery configuration and is configured to be implanted into a native leaflet of a native heart valve by distally advancing the pusher shaft when the catheter is in the implantation configuration.

This application claims the benefit of U.S. Provisional Patent Application Ser. No. 63/384,447, filed Nov. 21, 2022, the entire content of which is incorporated herein by reference.

### FIELD OF THE INVENTION

The present technology is generally related to the use of radiopaque markers to assist the placement of heart valve prostheses.

### BACKGROUND OF THE INVENTION

Patients suffering from various medical conditions or diseases may require surgery to install an implantable medical device. For example, valve regurgitation or stenotic calcification of leaflets of a heart valve may be treated with a heart valve replacement procedure. A traditional surgical valve replacement procedure requires a sternotomy and a cardiopulmonary bypass, which creates significant patient trauma and discomfort. Traditional surgical valve procedures may also require extensive recuperation times and may result in life-threatening complications.

One alternative to a traditional surgical valve replacement procedure is delivering implantable medical devices using minimally-invasive techniques. For example, a transcatheter heart valve prosthesis can be percutaneously and transluminally delivered to an implant location. In such methods, the transcatheter heart valve prosthesis can be compressed or crimped on a delivery catheter for insertion within a patient's vasculature; advanced to the implant location; and re-expanded to be deployed at the implant location.

For example, transcatheter aortic valve implantation (TAVI) is used to implant a transcatheter aortic valve (TAV) at the location of a native aortic valve. During a TAVI procedure, it is critical for the clinician to know the location of the native anatomy for proper positioning of the TAV. For example, and not by way of limitation, depth of the implant is crucial in TAVI procedures. If the TAV is too high, the TAV may migrate upwards (towards the aorta AO). If too low, the TAV can cause conduction disturbances, leading to permanent pacemaker implantation (PPI). Currently, during TAVI procedures, clinicians rely on injection of contrast solution and a pigtail catheter PC (see FIG. 1). The pigtail catheter PC is located in the nadir of the aortic valve AV leaflets LF and in combination with the contrast media assists in locating the proper depth for implanting the TAV. However, the use of contrast media can lead to a variety of complications, such as an increased risk of acute kidney injury. Therefore, there is a need for improved devices and methods for locating the native anatomy during a TAVI procedure such that the TAV may be properly located with minimal or no usage of contrast media.

### BRIEF SUMMARY OF THE INVENTION

The techniques of this disclosure generally relate to the use and implantation of radiopaque markers.

In a first example, the present disclosure is directed to a system for implanting a radiopaque marker including a catheter having a distal end and a proximal end, the catheter including a delivery configuration and an implantation configuration, the catheter further including: a first shaft configured to curl when the catheter is in the implantation configuration, the first shaft including a first opening positioned at a side of the first shaft; a second shaft positioned within the first shaft and configured to transition the catheter between the delivery configuration and the implantation configuration, the second shaft including a second opening; and a pusher shaft positioned within the second shaft; and a radiopaque marker disposed within the second shaft with the catheter in the delivery configuration, the radiopaque marker configured to be implanted into a native leaflet of a native heart valve; wherein the radiopaque marker is implanted by the pusher shaft when the catheter is in the implantation configuration.

In a second example, in the system according to any of the previous or subsequent examples herein, the at least two radiopaque markers are positioned within the second shaft.

In a third example, in the system according to any of the previous or subsequent examples herein, the second shaft moves proximally relative to the first shaft to transition the pigtail catheter from the delivery configuration to the implantation configuration.

In a fourth example, in the system according to any of the previous or subsequent examples herein, when the catheter is in the implantation configuration, the first opening and the second opening are axially aligned.

In a fifth example, in the system according to any of the previous or subsequent examples herein, the pusher shaft is configured to push the marker through the first opening and the second opening and to insert the marker into the native leaflet.

In a sixth example, in the system according to any of the previous or subsequent examples herein, the first shaft is formed of a shape memory material and is shape set to the implantation configuration.

In a seventh example, in the system according to any of the previous or subsequent examples herein, the second shaft is sufficient stiff such that with a distal end of the second shaft disposed adjacent a distal end of the first shaft, the catheter is in the delivery configuration and is substantially straight.

In an eighth example, in the system according to any of the previous or subsequent examples herein, retracting the second shaft relative to the first shaft enables the first shaft to return to is shape set configuration.

In a ninth example, in the system according to any of the previous or subsequent examples herein, the first shaft has a first stiffness and the second shaft has a second stiffness, wherein the second stiffness is greater than the first stiffness.

In a tenth example, in the system according to any of the previous or subsequent examples herein, the catheter further includes a handle at a proximal end thereof, wherein the handle is configured to control the transition between the delivery configuration and the implantation configuration, and control the pusher shaft to implant the radiopaque marker.

In an eleventh example, a radiopaque marker includes a body having a proximal end and a distal end, the body configured to be straight in a delivery configuration and the body configured to change to a predetermined coil or spiral shape in an implanted configuration. The radiopaque marker further includes a tip positioned at the distal end of the body and configured to pierce a native leaflet of a native heart valve, an a head positioned at the proximal end of the body, the head configured to rest on a surface of the native leaflet when implanted in the leaflet.

In a twelfth, in the radiopaque marker according to any of the previous or subsequent examples herein, the body is at least partially implanted within the native leaflet when the radiopaque marker is implanted in the leaflet.

In a thirteenth example, in the radiopaque marker according to any of the previous or subsequent examples herein, the body comprises a shape memory material shape set to the implantation configuration.

In a fourteenth example, a method for implanting a radiopaque marker includes delivering a catheter in a delivery configuration to a native leaflet of a native valve, the catheter including a first shaft having a first opening and a second shaft positioned within the first shaft and having a second opening, transitioning the catheter from the delivery configuration to an implantation configuration in which the first shaft is curved and the first opening faces the native leaflet, and implanting a radiopaque marker from the second shaft through the second opening and the first opening, and into the native leaflet.

In a fifteenth example, in the method according to any of the previous or subsequent examples herein, transitioning the catheter from the delivery configuration to the implantation configuration comprises retracting the second shaft relative to the first shaft.

In a sixteenth example, in the method according to any of the previous or subsequent examples herein, the catheter further includes a pusher shaft slidably disposed within the second shaft, wherein implanting the radiopaque marker comprises distally advancing a pusher shaft relative to the second shaft to push the radiopaque marker through the second opening and the first opening.

In a seventeenth example, in the method according to any of the previous or subsequent examples herein, the radiopaque marker is a first radiopaque marker and the native leaflet is a first native leaflet, wherein the method further comprises: transitioning the catheter back to the delivery configuration after implanting the first radiopaque marker in the first native leaflet; moving the catheter to a second leaflet of the native heat heart valve; transitioning the catheter to the implantation configuration from the delivery configuration; and implanting a second radiopaque marker from the second shaft through the second opening and the first opening, and into the second native leaflet.

### BRIEF DESCRIPTION OF DRAWINGS

The foregoing and other features and advantages of the invention will be apparent from the following description of embodiments thereof as illustrated in the accompanying drawings. The accompanying drawings, which are incorporated herein and form a part of the specification, further serve to explain the principles of the invention and to enable a person skilled in the art to make and use the invention. The drawings are not to scale.

FIG. 1 depicts a schematic representation of a native aortic valve with a pigtail catheter disposed therein.

FIG. 2 depicts a schematic cross-sectional side view of the distal end of a catheter for delivering radiopaque markers in a delivery configuration according to aspects of the disclosure.

FIG. 3 depicts a schematic cross-sectional side view of the distal end of the catheter of FIG. 2 in an implantation configuration according to aspects of the disclosure.

FIG. 4 depicts a schematic cross-sectional side view of the distal end of the catheter of FIG. 2 in an implantation configuration with a radiopaque marker being implanted therefrom according to aspects of the disclosure.

FIG. 5A depicts a schematic close-up view of an embodiment of the radiopaque markers disposed in a shaft according to aspects of the disclosure.

FIG. 5B depicts a schematic close-up view of an embodiment of the radiopaque markers disposed in a shaft according to aspects of the disclosure.

FIG. 6 depicts a schematic view of the radiopaque markers of FIG. 5 in a desired location of the heart according to aspects of the disclosure.

FIG. 7 depicts a schematic view of the radiopaque markers of FIG. 5 in a desired location of the heart after a prosthetic heart valve has been placed in a native valve according to aspects of the disclosure.

FIGS. 8A and 8B depict a schematic illustrations of another embodiment of a radiopaque marker according to aspects of the disclosure.

FIG. 9 depicts a schematic cross-sectional side view of the distal end of a catheter in a delivery configuration with the radiopaque marker of FIG. 8 disposed therein according to aspects of the disclosure.

FIG. 10 depicts a schematic cross-sectional side view of the distal end of the catheter of FIG. 9 in an implantation configuration with the radiopaque marker of FIG. 8 according to aspects of the disclosure.

FIG. 11 depicts a schematic cross-sectional side view of the distal end of the catheter of FIG. 9 in an implantation configuration with the marker of FIG. 8 being implanted according to aspects of the disclosure.

FIG. 12 depicts a view of a second embodiment of radiopaque markers in a desired location of the heart in accordance with an aspect of the disclosure.

FIG. 13 depicts a close-up of a second embodiment of the radiopaque markers in a desired location of the heart after a prosthetic heart valve has been placed in a native valve in accordance with an aspect of the disclosure.

FIG. 14 depicts a perspective view of a handle in a delivery configuration in accordance with an aspect of the disclosure.

FIG. 15 depicts a perspective view of a handle in an implantation configuration in accordance with an aspect of the disclosure.

FIG. 16 depicts a cross-sectional view of a handle in the implantation configuration in accordance with an aspect of the disclosure.

FIG. 17 depicts a close-up view of a click pen mechanism in accordance with an aspect of the disclosure.

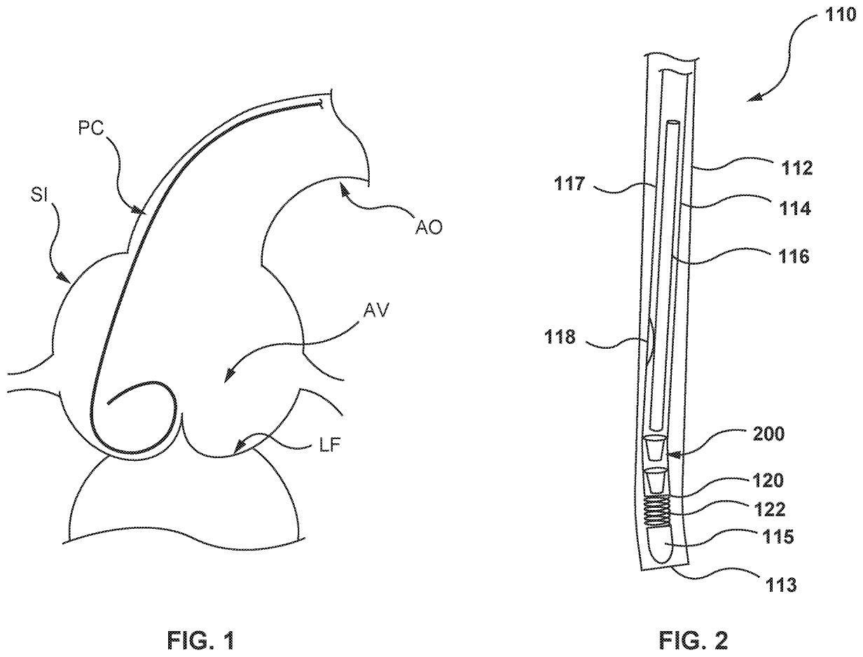

### DETAILED DESCRIPTION OF THE INVENTION

Specific embodiments of the present invention are not described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and use of the invention. Although the description of the invention is in the context of the treatment and navigation of a heart valve, the invention may be used where it is deemed useful in other anatomical sites that are not in the heart. For example, the present invention may be applied to other heart valves or venous valves as well. Furthermore, there is no intention to be bound by any expressed or implied theory presented in the preceding technical field, background, brief summary, or the following detailed description.

In some embodiments, a system 100 for implanting a radiopaque marker allows for a clinician to see the native geometry of the heart valve during placement of a heart valve prosthesis. The system generally includes a catheter 110, markers 200, and a handle 400. Broadly, the catheter 110 is placed within the nadir of the native cusp of a heart valve and implants radiopaque markers 200. In an embodiment, a marker 200 may be placed in each of the three native cusps. However, this is not meant to be limiting, and more or fewer markers 200 may be utilized. The use of three markers 200, with each in a nadir of one of the three native cusps provides information regarding the native anatomy in three-dimensional space (degree of parallax). Further, the approximate location of the coronaries relative to the inserted heart valve prosthesis may be provided, thereby reducing the risk of misplacing the heart valve prosthesis and blocking the coronary arteries. Further, the use of three markers 200 enables the clinician to determine the depth of the heart valve prosthesis versus the native anatomy on all sides of the heart valve prosthesis.

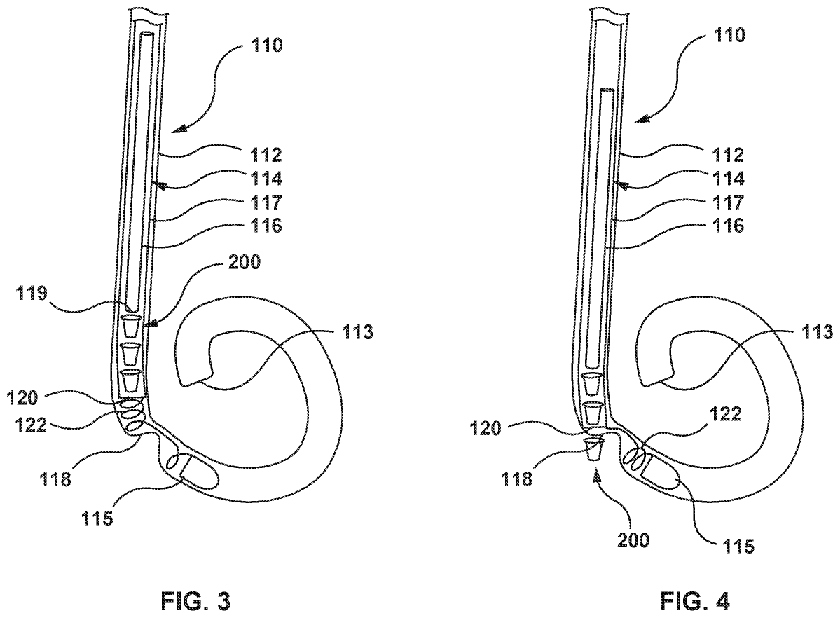

The catheter 110 is used to navigate the human anatomy and implant markers 200, and generally includes a flexible shaft 112, a stiff shaft 114, a pushing shaft 116, and a valve 124. When the catheter 110 is in a delivery configuration, as shown in FIG. 2, the catheter 110 is capable of being controlled by a clinician to be navigated, or tracked, through a patient's body to be positioned in the native heart cusps. The catheter 110 may be delivered to the heart via percutaneous transfemoral, transradial, transaortic, transcaval, transcarotid, or transaxillary approach, and may be positioned within the desired area of the heart via different delivery methods known in the art for accessing heart valves. Once delivered to the native heart cusp, the catheter 110 transitions to the implantation configuration, as shown in FIG. 3, allowing for a clinician to implant the radiopaque markers 200. The catheter is designed to curl into a shape that sits into the cusp, using shapes that are familiar to the clinician, allowing the clinician to determine when they are in contact with the annulus. More detail regarding the components will be provided herein.

Referring to FIG. 2, the flexible shaft 112 is a hollow tube that connects to and extends away from the handle 400 and results in an enclosed tip at a distal end 113 thereof. To enable the catheter 110 to curl or “pigtail”, the distal end of the flexible shaft 112 may have a pre-determined shape such as a fixed angle, slight angle, cobra, visceral, or pigtail. For example, the distal portion may be formed of a shape memory material that forms the curved “pigtail” shape unless restrained by a force, such as by the stiff shaft 114 described herein. Further, the flexible shaft 112 is in contact with native tissue and is exposed to bodily fluids, therefore, the flexible shaft 112 may be made of a bio-compatible material such as polyether block amide or polyamide blends. The flexible shaft 112 further includes an opening 118 disposed proximal of the distal end 113 of the flexible shaft 112. The opening 118 is configured to open when the flexible shaft 112 is in the implantation configuration and is located such that with the flexible shaft 112 in the implantation configuration, the opening 118 enables the markers 200 to exit the flexible shaft 112 through the opening 118, as explained in more detail below. In the embodiment shown, the opening 118 is a side opening in the flexible shaft 112 that is subsequently directed distally when the flexible shaft 112 is in the implantation configuration, as described below.

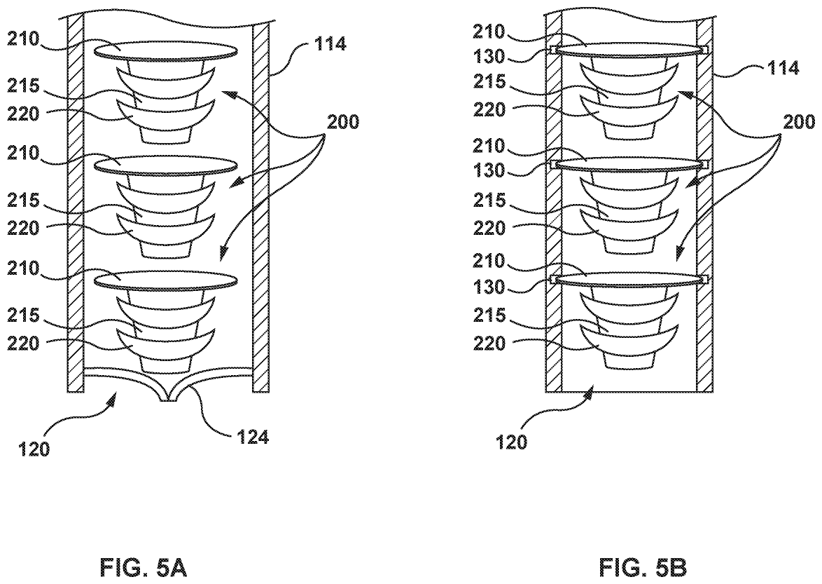

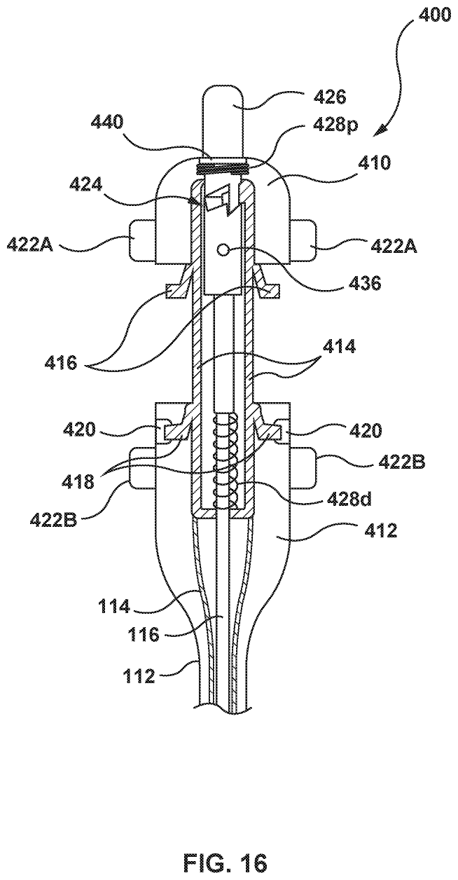

The stiff shaft 114 is a hollow shaft that is positioned within the flexible shaft 112 and extends the length of the flexible shaft 112, and includes a shaft portion 117, a distal tip 115, and a coil 122 coupling the shaft portion 117 to the distal tip 115. A proximal end of the shaft portion 117 is coupled to the handle (see FIG. 16) enabling the clinician to move the stiff shaft 114 proximally and distally relative to the flexible shaft 112. When the distal tip 115 of the stiff shaft 114 is positioned at the distal end 113 of the flexible shaft 112, as seen in FIG. 2, the stiff shaft 114 will hold the flexible shaft 112 substantially straight (i.e., not in the curved or pigtail configuration). In other words, the stiff shaft 114 will overcome the shape memory of the flexible shaft 112. However, as the stiff shaft 114 moves proximally relative to the distal end 113 of the flexible shaft 112, the flexible shaft 112 will begin to revert to the predetermined shape, as shown in FIG. 3. The shaft portion 117 of the stiff shaft 114 is configured to hold the markers 200 within the central passageway or lumen of the shaft portion 117. The shaft portion 117 of the stiff shaft 114 includes an opening 120 adjacent a distal end thereof and may also include a valve 124 or similar device (see FIG. 5A) disposed adjacent the opening 120 to prevent the markers 200 from escaping through the opening 120 until the clinician initiates deployment of the markers 200, as explained in more detail below.

FIG. 3 shows the implantation configuration with the stiff shaft 114 moved proximally relative to the flexible shaft 112 such that the flexible shaft 112 is in the curved or pigtail shape. Further, in the implantation configuration of FIG. 3, the opening 120 of stiff shaft 114 is disposed proximal of the opening 118 of the flexible shaft 112 such that the markers 200 may exit the opening 120 and the opening 118, as explained below. Although FIG. 3 shows the implantation configuration with the opening 120 proximal of the opening 118, the stiff shaft 114 may be retracted lesser amounts relative to the distal end 113 of the flexible shaft 112 to impart different degrees of curves or bends to the flexible shaft 112, such as for steering.

The coil 122 prevents the opening 118 in the flexible shaft 112 from being blocked when the flexible shaft 112 is in the implantation configuration, as shown in FIG. 3. In particular, as shown in FIG. 3, when in the implantation configuration, the distal tip 115 of the stiff shaft 114 is positioned distal of the opening 118 in the flexible shaft 112 and the shaft portion 117 is disposed proximal of the opening 118, with the coil 122 extending therebetween. This also prevents the shaft portion 117 of the stiff shaft 114 from catching on an edge of the opening 118 when the stiff shaft 114 is translated distally to re-straighten the flexible shaft 112, such as for re-positioning and/or withdrawing the catheter 110.

As noted above, shaft portion 117 of the stiff shaft 114 may further include the valve 124 disposed adjacent the opening 120, as shown in FIG. 5A. The valve 124 retains the markers 200 within the shaft portion 117 of the stiff shaft 114 when the catheter 110 is in the implantation configuration. As described in more detail below, when the markers 200 are to be implanted, the force of the pusher shaft 116 on the markers 200 is transferred to the valve 124 with sufficient force to overcome the valve 124, thereby opening the valve 124. Once the markers 200 have been implanted and/or after each individual marker 200 has been implanted, the valve 124 closes, and, in some embodiments, retains additional markers 200 within the shaft portion 117 of the stiff shaft 114. Although a valve with flaps is shown, this is not meant to be limiting, and other embodiments for retaining the markers 200 in the shaft portion 117 may be utilized. For example, and not by way of limitation, grooves 130, as shown in FIG. 5B, may be provided in the inner surface of the shaft portion 117 of the stiff shaft 114. A portion of each marker 200, such as a head 210 of each marker, is partially disposed within a respective groove 130, thereby retaining the markers 200 in the shaft portion 117 until the pusher shaft 116 pushes the markers 200, thereby overcoming the retaining force of the grooves 130.

The pusher shaft 116 may be a solid shaft positioned within the central passageway of the stiff shaft 114 and is configured to implant the markers 200 within the native cusps. The pusher shaft 116 is slidably movable relative to both the flexible shaft 112 and the stiff shaft 114. The proximal end of the pusher shaft 116 is operatively coupled to the handle 400 (see FIG. 16), enabling a clinician to control the movement of the pusher shaft 116, while a distal end 119 of the pusher shaft 116 is configured to place a sufficient force as to dispel the markers 200 out of the catheter 110 and into the native cusps. Thus, the distal end 119 of the pusher shaft 116 is disposed within the stiff shaft 114 proximal to the markers 200, as shown in FIGS. 2 and 3. As explained in more detail below, movement of the pusher shaft 116 may be limited to a predetermined distance to ensure that only one marker 200 is pushed out of the catheter 110 at a time.

An embodiment of the markers 200 is shown in FIGS. 5A and 5B. In this embodiment, each marker includes a proximal head 210 and a body 215 extending distally from the head 210. The body 215 may taper in the distal direction such that a distal end of the body has a smaller diameter than a proximal end of the body 215. The taper allows for the body 215 to be easily implanted into the native tissue. In some embodiments, the body 215 is intended to not pierce entirely through the cusp but to traverse through only a portion of the cusp. The head 210 is configured to receive a force from the pusher shaft 116 and may be a flat or substantially flat surface positioned at the proximal end of the marker 200. The diameter of the head 210 is generally larger than the diameter of the body 215, allowing for the head 210 to restrict the depth the marker 200 is implanted into the native tissue. Once implanted, the head 210 of the marker 200 may rest on the surface of the native tissue while the body 215 is positioned within the tissue. In some embodiments where multiple markers 200 are utilized, the head 210 of each marker 200 may be shaped differently, allowing for the clinician to differentiate between each of the implanted markers 200. For example, and not by way of limitation, the head 210 of a first marker may be circular, the head 210 of a second marker 200 may be rectangular, and the head 210 of the third marker 200 may be triangular. The head 210 generally has a diameter or transverse dimension between 0.5 mm-2 mm, however, this range can be expanded to adapt the markers 200 to different specified uses. The body 215 and the head 210 may be manufactured as a singular piece or may be manufactured separately and coupled together using methods known in the art.

In some embodiments, each marker 200 further includes at least one barb 230 extending outwardly from the body 215. As seen in FIGS. 5A and 5B, each marker 200 may include two barbs 220, but that is not meant to be limiting, and more or fewer barbs 220 may be utilized. The barbs 220 may be curved or angled proximally towards the head 210, as shown, enabling both easier implantation and additional securement/prevention of the marker 200 backing out of the tissue. The barbs 220 may be manufactured as a singular piece as the body 215 or may be manufactured separately and coupled to the body 215 using methods known in the art.

The markers 200 are radiopaque. The markers 200 may be made with radiopaque material or coated, infused, or otherwise include a radiopaque material. The entirety of each marker 200 may be radiopaque, or portions of the markers 200, such as the head 210, may be radiopaque. Examples of radiopaque material that may be used for parts or all of each marker 200 include, but are not limited to, stainless steel or gold.

In order to implant the markers 200, the catheter 110 is delivered to the heart of the patient. The catheter 110 may be delivered using routes through the vasculature as known to those skilled in the art, such as but not limited to, transfemoral access to a femoral artery to an iliac artery to the aorta and over the aortic arch, or transradial access to a radial artery to a brachial artery to a subclavian artery and into the aortic arch through the brachiocephalic artery. The catheter 110 may be delivered within a guide tube as known to those skilled in the art. The catheter 110 may be delivered similar to traditional pigtail catheters for injecting contrast dye at the site of a native heart valve, as known to those skilled in the art.

Once at the site of native heart, the clinician will align the catheter 110 with one of the cusps of the native heart valve. The catheter 110 may then be transitioned from the delivery configuration shown in FIG. 2 to the implantation configuration shown in FIG. 3 by proximally retracting the stiff shaft 114 with the pusher shaft 116 and the markers 200 disposed therein such that the opening 120 of the stiff shaft 114 is proximal to the opening 118 of the flexible shaft 112. This enables the flexible shaft 112 to return to its predetermined shape, that is, the curved or pigtail shape shown in FIG. 3. In this implantation configuration, the opening 120 of the shaft portion 117 of the stiff shaft 114 is aligned with the opening 118 of the flexible shaft 112, as shown in FIG. 3.

With the catheter 110 aligned with a nadir of one of the cusps of the native heart valve and in the implantation configuration, the pusher shaft 116 may be advanced distally, thereby applying a distally directed force on the markers 210. This distally directed force is sufficient to overcome the retaining force of the valve 124 or the grooves 130 or other such retaining force, thereby ejecting the distal most marker 200 through the opening 120, through the opening 118 (as shown in FIG. 4), and into the native tissue of the cusp. As noted above, ideally the marker will be placed in the nadir of the cusp.

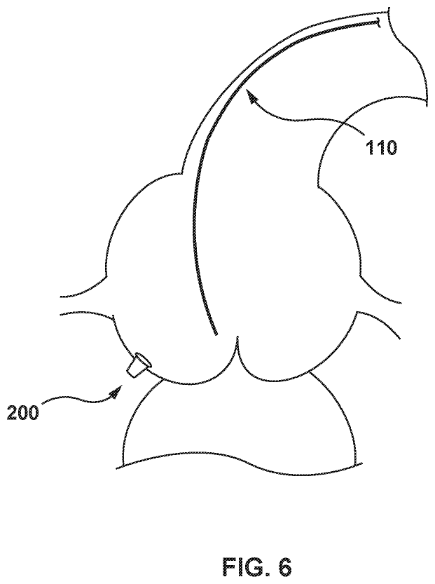

In some embodiments, as discussed above, it is desirable to implant a marker in each of the native cusps. In such an embodiment, a plurality of markers 200 may be positioned within the catheter 110, specifically within the shaft portion 117 of the stiff shaft 114. After implanting the distal-most marker 200 into a first cusp of native valve, the stiff shaft 114 may be advanced distally to transition the catheter from the implantation configuration to the delivery configuration, as shown in FIG. 6. The catheter 110 may then be moved to a second cusp of the native valve, and the process described above for implanting the marker may be repeated. In embodiments, such as implanting a marker 200 in each of the three native cusps of a native aortic valve, the catheter 110 includes three markers 200 that are implanted sequentially as discussed above. However, in some embodiments, only a single marker 200 will be implanted. After the final marker 200 is implanted, the catheter 110 is transitioned back the delivery configuration by advancing the stiff shaft 114 distally, and the catheter 100 can then be removed from the patient, or can remain in the patient during a subsequent procedure similar to prior pigtail catheters.

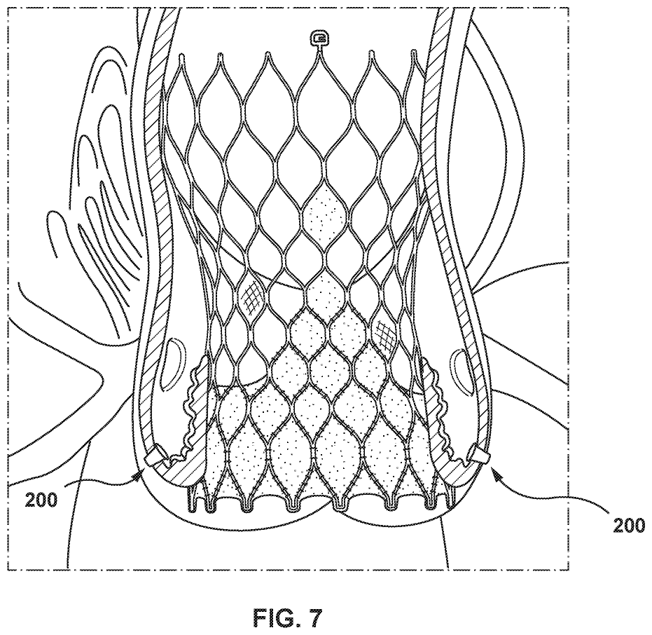

As explained above, the markers 200 may assisted in properly positioning a heart valve prosthesis within the native valve. The markers 200 may remain implanted within the native cusps after implantation of the heart valve prosthesis, as shown in FIG. 7.

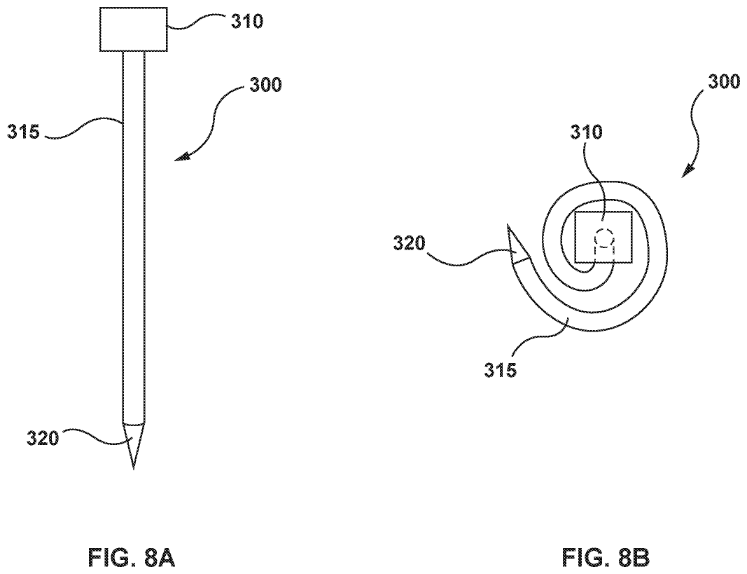

FIGS. 8A and 8B show another embodiment of a radiopaque marker 300 that can be used in aspects of the present disclosure. The marker 300 of FIGS. 8A and 8B includes a head 310, an elongate body 315, and a tip 320. FIG. 8A shows the marker 300 in a delivery configuration with the elongate body 315 straightened, and FIG. 8B shows the marker 300 in a deployed configuration with the elongate body 315 coiled or helical shape. In embodiments, the coiled or helical shape is a flat coil or helix. The elongate body 315 is formed of a shape memory material such that the marker 300 is in the deployed configuration unless a force is applied to it to straighten the elongate body into the delivery configuration, as explained below. The tip 320 of the marker is a sharp tip configured to penetrate tissue.

The marker 300 is radiopaque. The marker 300 may be made with radiopaque material or coated, infused, or otherwise include a radiopaque material. The entirety of each marker 300 may be radiopaque, or portions of each marker 300, such as the head 310, may be radiopaque. Examples of radiopaque material that may be used for parts or all of each marker 300 include, but are not limited to, stainless steel or gold.

The head 310 is configured to receive a force from the pusher shaft 116 and may be a flat or substantially flat surface positioned at the proximal end of the marker 300. The diameter of the head 310 is generally larger than the diameter of the elongate body 315, allowing for the head 310 to serve as a stop for implantation of the marker 300 into the native tissue. Once implanted, the head 310 of the marker 200 may rest on the surface of the native tissue while the body 315 is positioned within the tissue. In some embodiments where multiple markers 300 are utilized, the head 310 of each marker 300 may be shaped differently, allowing for the clinician to differentiate between each of the implanted markers 300. For example, and not by way of limitation, the head 310 of a first marker may be circular, the head 310 of a second marker 300 may be rectangular, and the head 310 of the third marker 300 may be triangular. The head 310 generally has a diameter or transverse dimension between 0.5 mm-2 mm, however, this range can be expanded to adapt the markers 300 to different specified uses. The body 315, the head 310, and the tip 320 may be manufactured as a singular piece or may be manufactured separately and coupled together using methods known in the art.

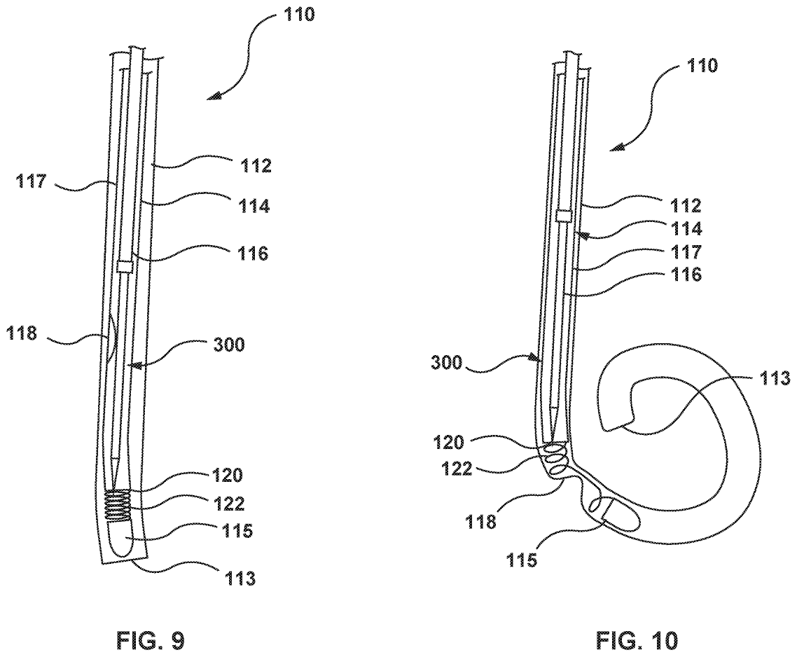

FIGS. 9-11 show the marker 300 disposed in the catheter 110 described above. The catheter 110 shown in FIGS. 9-11 is substantially the same as the catheter 110 described above. Thus, it will not be described again. FIG. 9 shows the catheter 100 in the delivery configuration with the distal tip 115 of the stiff shaft 114 adjacent the distal end 113 of the flexible shaft 112. The marker 300 is disposed within the shaft portion 117 of the stiff shaft 114. The shaft portion 117 of the stiff shaft 114 maintains the marker 300 in the delivery configuration, such that the elongate body 315 is straightened.

FIG. 10 shows the catheter 110 in the implantation configuration with the stiff shaft 114 retracted proximally relative to the flexible shaft 112 such that the flexible shaft 112 assumes pigtail or curved state. As described above, the opening 120 in the shaft portion 117 of the stiff shaft 114 is aligned with the opening 118 in the flexible shaft 112 such that the marker 300 is ready to be implanted.

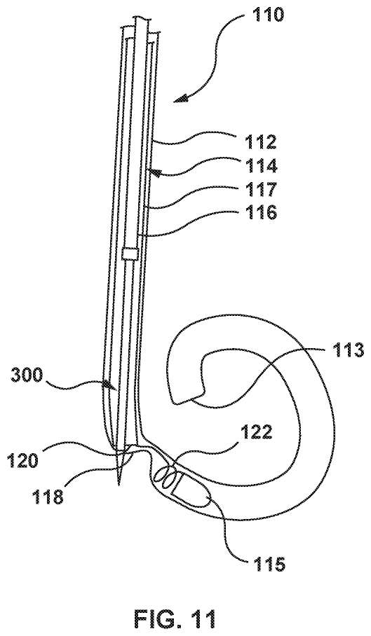

As discussed above, the catheter 110 is aligned with a native cusp such that the opening 118 is adjacent the native tissue of the cusp. FIG. 11 shows the pusher shaft 116 being pushed distally, which pushes the head 310, and thus the marker 300 distally. As the tip 320 of the marker 300 exits the opening 118, the tip 320 will pierce the tissue of the cusp. Further, as the marker continues to be pushed out of the opening 118, the elongate body 315/tip 320 will no longer be restrained by the shaft portion 117 of the stiff shaft 114. Therefore, the body 315/tip 320 will revert to the coiled configuration as they exit the opening 118. This will cause the body 315/tip 320 to coil within the tissue of the cusp, thereby securing the marker to the cusp. The head 310 of the marker 300 will not pierce the tissue, indicating to the clinician that the marker 300 is implanted. In some embodiments, the body 315/tip 320 of the marker 300 do not extend entirely through the cusp. In other embodiments, at least a portion of the body 315/tip 320 extends entirely through the cusp.

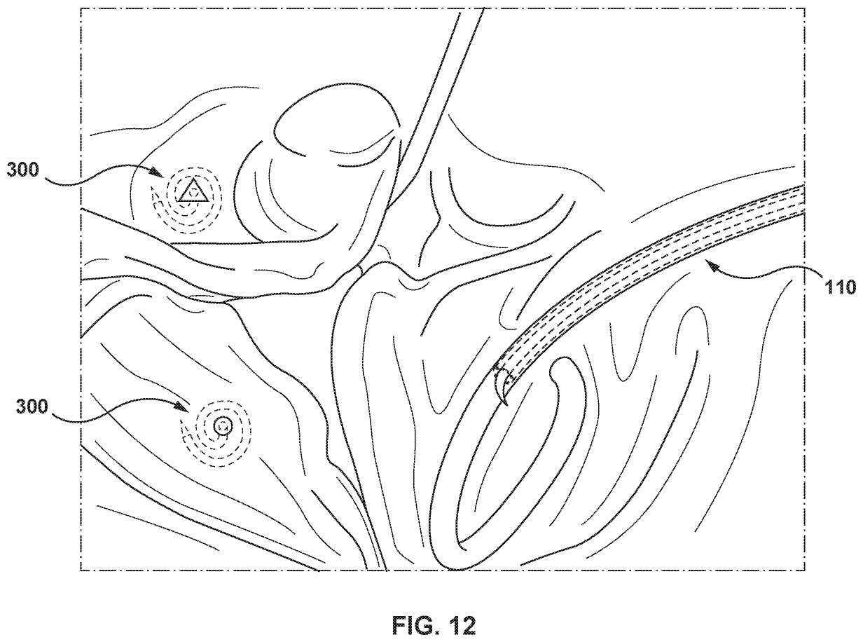

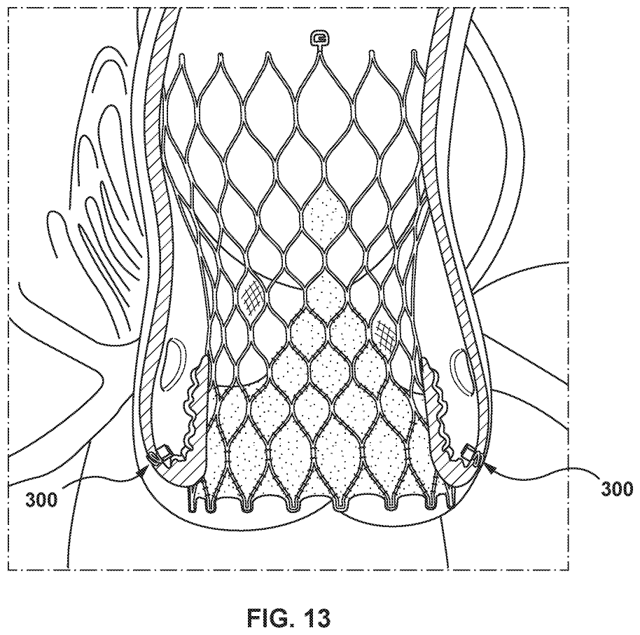

FIGS. 12 and 13 depict multiple markers 300 implanted in a corresponding cusp, with FIG. 13 showing the markers 300 after implantation of a heart valve prosthesis. Multiple markers 300 may be implanted as described above in a single catheter 110, or multiple catheters 110 may be utilized.

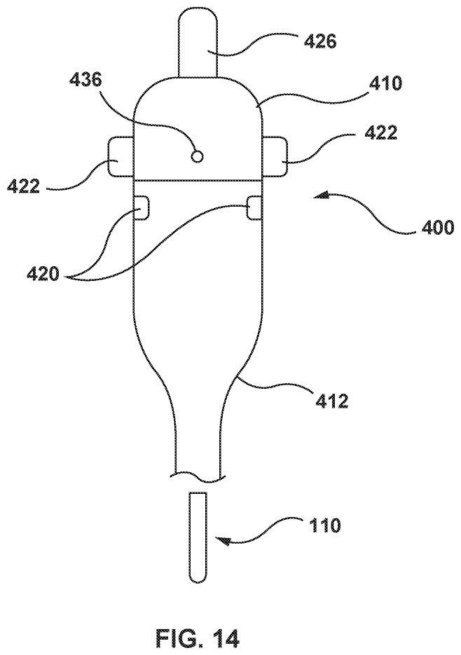

FIGS. 14-17 depict a handle 400 of the catheter 110. The handle 400 is an example only and other handles 400 may also be utilized with the catheter 110 to deploy the markers 200, 300. The handle 400 is positioned at the proximal end of the catheter 110, controls the transition of the catheter 110 from the straight delivery configuration to the implantation configuration, and controls implantation of the markers 200/300 into the native tissue. The handle 400 generally includes a proximal handle portion 410 and a distal handle portion 412.

The handle 400 is operatively connected to the flexible shaft 112, stiff shaft 114, and the pusher shaft 116, controlling the movement of each of the shafts. In an embodiment, the distal handle portion 412 is connected to the flexible shaft 112, while the stiff shaft 114 is connected to the proximal handle portion 410. Therefore, the proximal handle portion 410 can be moved relative to the distal handle portion 412 to move the stiff shaft 114 relative to the flexible shaft 112. As described above, movement of the stiff shaft 114 relative to the flexible shaft 114 transitions the catheter 110 between the delivery configuration and the implantation configuration. Those skilled in the art would recognize that other handles may be utilized, such as handles with the flexible shaft fixedly attached to the handle and the stiff shaft coupled to an actuator of the handle that can be moved relative to the handle, thereby moving the stiff shaft relative to the flexible shaft.

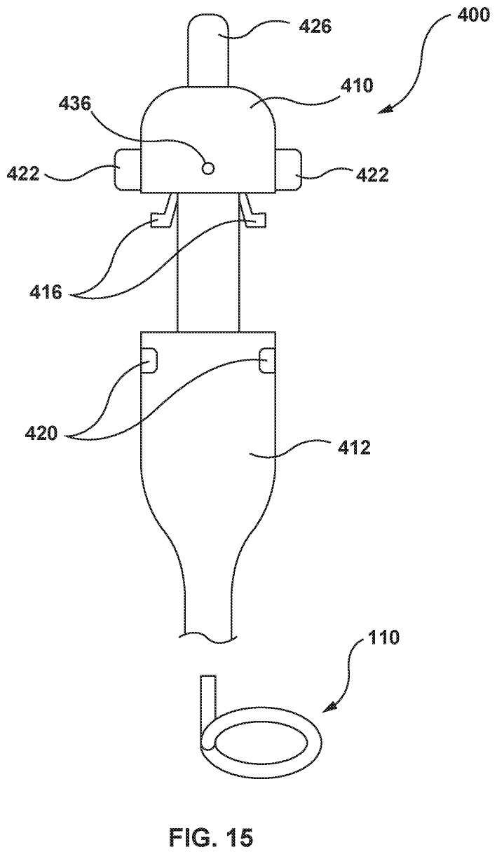

In the embodiment shown, the handle 400 further includes a first snap position, shown in FIG. 14, and a second snap position, shown in FIG. 15, that locks the handle in either the delivery configuration or the implantation configuration. To accomplish this, as shown in FIG. 16, the handle 400 includes a snap body structure 414 that includes proximal tabs 416 extending radially outwardly and distal tabs 418 extending radially outward. The tabs 416, 418 are configured to engage with corresponding of mating holes or grooves 420 in the distal handle portion 412 of the handle 400. The delivery configuration correlates to when the distal handle portion 412 of the handle is adjacent to the proximal handle portion 410 such that the proximal tabs 416 engage the mating holes 420, as shown in FIG. 14. The implantation configuration correlates to when the distal handle portion 410 is spaced from the proximal handle portion 412 such that the distal tabs 418 engage the mating holes 420, as shown in FIGS. 15 and 16. In the embodiment shown, the proximal and distal tabs 416, 418 are circumferentially aligned with each other, i.e., each of the proximal tabs 416 is on the same longitudinal axis as a corresponding one of the distal tabs 418. However, this is not meant to be limiting. For example, in other embodiments, the proximal tabs may be circumferentially offset from the distal tabs 418 by 90 degrees. In such, an embodiment, the opening 120 of the stiff shaft 114 and the opening 118 of the flexible shaft 112 are misaligned in the delivery configuration, thereby providing greater structural integrity during delivery of the catheter 110 through the vasculature.

In the embodiment shown, the handle 400 will remain in either the first snap position or the second snap position until actively released therefrom. In an embodiment, the handle 400 includes a lock release 422 to release the handle from the first snap position and the second snap position, as shown in FIGS. 14 and 15. In another embodiment, shown in FIG. 16, the handle 400 includes a proximal lock release 422A to release the handle from the first snap position and a distal lock release 422B to release the handle from the second snap position. In the embodiments shown, the lock releases 422, 422A, 422B comprise buttons or tabs on opposite sides of the proximal portion 410 and/or the distal portion 412 of the handle 400. Squeezing the buttons pushes the snap body structure 414 radially inward, thereby pushing the proximal and distal tabs 416, 418 radially inward and out of the mating holes 420. With the proximal and distal tabs 416, 418 released from the mating holes 420, the clinician may slide the proximal portion 410 relative to the distal portion 420.

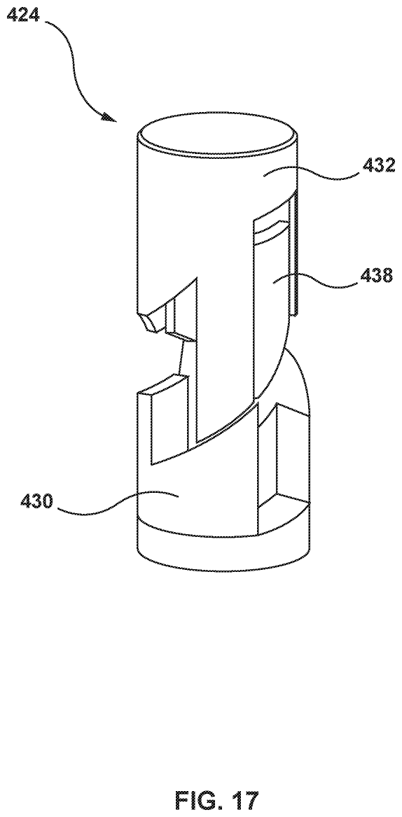

In the embodiment shown, the handle 400 is configured to deploy and implant the markers 200/300 using a click pen mechanism 424 that is coupled to a push button 426 and the pusher shaft 116, as shown in FIGS. 16 and 17. When the button 426 is depressed by a clinician, the click pen mechanism 424 forces the pusher shaft 116 distally, subsequently pushing on the marker 200/300 distally out of the stiff shaft 114 and the flexible shaft 112, and into the native cusp. Upon release of the click pen mechanism, a distal spring 428d positioned within the snap body structure 414 adjacent a distal end of the snap body structure 414, and a proximal spring 428p disposed between a proximal end of the snap body structure 414 and a lip 440 of the button 426 return the button 426, the click pen mechanism 424, and the pusher shaft 116 back to the starting position.

The click pen mechanism 424 is shown in FIG. 17 and generally includes a cam body 430, a tubular plunger 432, and a series of fixed stop members 434. When the button 426 is depressed, the button 426 pushes the tubular plunger 432 distally, which rotates and pushes the cam body 430 distal of the series of stop members 438. At this point, the pusher shaft 116 will be depressed by the cam body 430, dispensing and implanting the marker 200/300. When the button 426 is released, the springs 428p/428d force the tubular plunger 432 back proximally, causing the cam body 430 to rotate again and strike the series of fixed stop members 438. The pusher shaft 116 will remain extended until the button 426 is pressed again, wherein the process described above repeats, ending with the pusher shaft 116 being retracted and returning to the default state. In some embodiments, a plurality of markers 200/300 are positioned within the catheter 110, and in order to deploy only a single marker 200/300 at a time, the click pen mechanism 424 will have varying heights. More specifically, the varying heights will correlate to how far the pusher shaft 116 is extended, which allows the clinician to only dispense a single marker 200/300 at a time.

In some embodiments, a lock pin mechanism 436 may be used to lock the click pen mechanism 424 so that a marker 200/300 cannot accidently be dispensed out of the catheter 110. The lock pin mechanism 436 may be enabled or disabled using a pin or a tab.

While various embodiments according to the present invention have been described above, it should be understood that they have been presented by way of illustration and example only, and not limitation. It will be apparent to a person skilled in the relevant art that various changes in form and detail can be made therein without departing from the spirit and scope of the invention. Thus, the breadth and scope of the present invention should not be limited by any one of each embodiment discussed herein, and of each reference cited herein, can be used in combination with the features of any other embodiment. All patents and publication discussed herein are incorporated by reference herein in their entirety.

The following examples are a non-limiting list of clauses in accordance with one or more techniques of this disclosure.
Example 1. A system for implanting a radiopaque marker comprising: a catheter having a distal end and a proximal end, the catheter including a delivery configuration and an implantation configuration, the catheter further including: a first shaft configured to curl when the catheter is in the implantation configuration, the first shaft including a first opening positioned at a side of the first shaft; a second shaft positioned within the first shaft and configured to transition the catheter between the delivery configuration and the implantation configuration, the second shaft including a second opening; and a pusher shaft positioned within the second shaft; and a radiopaque marker disposed within the second shaft with the catheter in the delivery configuration, the radiopaque marker configured to be implanted into a native leaflet of a native heart valve; wherein the radiopaque marker is implanted by the pusher shaft when the catheter is in the implantation configuration.
Example 2. The system of Example 1, wherein at least two radiopaque markers are positioned within the second shaft.
Example 3. The system of Example 1, wherein the second shaft moves proximally relative to the first shaft to transition the pigtail catheter from the delivery configuration to the implantation configuration.
Example 4. The system of Example 1, wherein, when the catheter is in the implantation configuration, the first opening and the second opening are axially aligned.
Example 5. The system of Example 5, wherein the pusher shaft is configured to push the marker through the first opening and the second opening and to insert the marker into the native leaflet.
Example 6. The system of Example 1, wherein the first shaft is formed of a shape memory material and is shape set to the implantation configuration.
Example 7. The system of Example 6, wherein the second shaft is sufficient stiff such that with a distal end of the second shaft disposed adjacent a distal end of the first shaft, the catheter is in the delivery configuration and is substantially straight.
Example 8. The system of Example 6, wherein retracting the second shaft relative to the first shaft enables the first shaft to return to is shape set configuration.
Example 9. The system of Example 7, wherein the first shaft has a first stiffness and the second shaft has a second stiffness, wherein the second stiffness is greater than the first stiffness.
Example 10. The system of Example 1, wherein the catheter further includes a handle at a proximal end thereof, wherein the handle is configured to control the transition between the delivery configuration and the implantation configuration, and control the pusher shaft to implant the radiopaque marker.
Example 11. A radiopaque marker having a delivery configuration and an implanted configuration comprising: a body having a proximal end and a distal end, the body configured to be straight in the delivery configuration and the body configured to change to a predetermined coil or spiral shape in the implanted configuration; a tip positioned at the distal end of the body and configured to pierce a native leaflet of a native heart valve; and a head positioned at the proximal end of the body, the head configured to rest on a surface of the native leaflet when implanted in the leaflet.
Example 12. The radiopaque marker of Example 11, wherein the body is at least partially implanted within the native leaflet when the radiopaque marker is implanted in the leaflet.
Example 13. The radiopaque marker of Example 10, wherein the body comprises a shape memory material shape set to the implantation configuration.
Example 14. A method for implanting a radiopaque marker comprising: delivering a catheter in a delivery configuration to a native leaflet of a native valve, the catheter including a first shaft having a first opening and a second shaft positioned within the first shaft and having a second opening; transitioning the catheter from the delivery configuration to an implantation configuration in which the first shaft is curved and the first opening faces the native leaflet; and implanting a radiopaque marker from the second shaft through the second opening and the first opening, and into the native leaflet.
Example 15. The method of Example 14, wherein transitioning the catheter from the delivery configuration to the implantation configuration comprises retracting the second shaft relative to the first shaft.
Example 16. The method of Example 14 or Example 15, wherein the catheter further includes a pusher shaft slidably disposed within the second shaft, wherein implanting the radiopaque marker comprises distally advancing a pusher shaft relative to the second shaft to push the radiopaque marker through the second opening and the first opening.
Example 17. The method of any one of Examples 14 to 16, wherein the radiopaque marker is a first radiopaque marker and the native leaflet is a first native leaflet, wherein the method further comprises: transitioning the catheter back to the delivery configuration after implanting the first radiopaque marker in the first native leaflet; moving the catheter to a second leaflet of the native heat heart valve; transitioning the catheter to the implantation configuration from the delivery configuration; and implanting a second radiopaque marker from the second shaft through the second opening and the first opening, and into the second native leaflet.

What is claimed is:

1. A system for implanting a radiopaque marker comprising:
a catheter having a distal end and a proximal end, the catheter including a delivery configuration and an implantation configuration, the catheter further including:
a first shaft configured to curl when the catheter is in the implantation configuration, the first shaft including a first opening positioned at a side of the first shaft;
a second shaft positioned within the first shaft and configured to transition the catheter between the delivery configuration and the implantation configuration, the second shaft including a second opening; and
a pusher shaft positioned within the second shaft; and
a radiopaque marker disposed within the second shaft with the catheter in the delivery configuration, the radiopaque marker configured to be implanted into a native leaflet of a native heart valve;
wherein the radiopaque marker is implanted by the pusher shaft when the catheter is in the implantation configuration.

2. The system of claim 1, wherein at least two radiopaque markers are positioned within the second shaft.

3. The system of claim 1, wherein the second shaft moves proximally relative to the first shaft to transition the pigtail catheter from the delivery configuration to the implantation configuration.

4. The system of claim 1, wherein, when the catheter is in the implantation configuration, the first opening and the second opening are axially aligned.

5. The system of claim 5, wherein the pusher shaft is configured to push the marker through the first opening and the second opening and to insert the marker into the native leaflet.

6. The system of claim 1, wherein the first shaft is formed of a shape memory material and is shape set to the implantation configuration.

7. The system of claim 6, wherein the second shaft is sufficient stiff such that with a distal end of the second shaft disposed adjacent a distal end of the first shaft, the catheter is in the delivery configuration and is substantially straight.

8. The system of claim 6, wherein retracting the second shaft relative to the first shaft enables the first shaft to return to is shape set configuration.

9. The system of claim 7, wherein the first shaft has a first stiffness and the second shaft has a second stiffness, wherein the second stiffness is greater than the first stiffness.

10. The system of claim 1, wherein the catheter further includes a handle at a proximal end thereof, wherein the handle is configured to control the transition between the delivery configuration and the implantation configuration, and control the pusher shaft to implant the radiopaque marker.

11. A radiopaque marker having a delivery configuration and an implanted configuration comprising:
a body having a proximal end and a distal end, the body configured to be straight in the delivery configuration and the body configured to change to a predetermined coil or spiral shape in the implanted configuration;
a tip positioned at the distal end of the body and configured to pierce a native leaflet of a native heart valve; and
a head positioned at the proximal end of the body, the head configured to rest on a surface of the native leaflet when implanted in the leaflet.

12. The radiopaque marker of claim 11, wherein the body is at least partially implanted within the native leaflet when the radiopaque marker is implanted in the leaflet.

13. The radiopaque marker of claim 10, wherein the body comprises a shape memory material shape set to the implantation configuration.
